(19) United States
(12) Patent Application Publication
(10) Pub. No.: US 2011/0313227 A1
(43) Pub. Date: Dec. 22, 2011

(54) ALKYLATION CATALYST AND RELATED PROCESS

(75) Inventors: Emanuel Hermanus van Broekhoven, Monnickendam (NL); Kar-Ming Au-Yeung, Amsterdam (NL)

(73) Assignee: Albemarle Europe SPRL, Louvain-la-Neuve (BE)

(21) Appl. No.: 13/148,447

(22) PCT Filed: Feb. 9, 2010
(86) PCT No.: PCT/EP2010/051595

§ 371 Date: Aug. 8, 2011

Related U.S. Application Data
(60) Provisional application No. 61/151,572, filed on Feb. 11, 2009.

(51) Int. Cl. C07C 2/58; B01J 29/04; B01J 29/08; B01J 29/00

(52) U.S. Cl. 585/722; 502/300; 502/60; 502/79; 502/344; 502/325; 502/339; 502/355

(57) ABSTRACT

A solid alkylation catalyst having a hydrogenation metal and a solid acid in the form of a rare earth exchanged molecular sieve, wherein the catalyst is at least characterized by a porosity of less than 0.20 ml/g in pores below 100 nm in diameter, and a total porosity of greater than 0.30 ml/g. A process for alkylation using the catalyst is also described.

As used herein, the term alkylation refers to the reaction of an alkylatable compound, e.g., a saturated hydrocarbon, with an alkylation agent, e.g., an olefin. The reaction is of interest because, e.g., it makes it possible to obtain through the alkylation of isobutane with an olefin containing 2-6 carbon atoms, an alkylate which has a high octane number and which boils in the gasoline range. Unlike gasoline obtained by cracking heavier petroleum fractions such as vacuum gas oil and atmospheric residue, gasoline obtained by alkylation is essentially free of contaminants such as sulfur and nitrogen and thus has clean burning characteristics. Its high anti-knock properties, represented by the high octane number, lessen the need to add environmentally harmful anti-knock compounds such as aromatics or lead. Also, unlike gasoline obtained by reforming naphtha or by cracking heavier petroleum fractions, alkylate contains few if any aromatics or olefins, which offers further environmental advantages.

Historically the activity and stability of solid acid alkylation catalysts have left much still to be desired when compared to competitive liquid acid alkylation processes. Recent developments in solid acid alkylation have included alkylation processes employing the facile regeneration of zeolite-containing solid acid catalysts, as disclosed in WO/9823560 (U.S. Pat. No. 5,986,158), improved solid acid catalyst production processes as per US Patent Application Publication 2007/0293390, alkylation catalyst hydration processes as per WO 2005/075387, continuous or semi-continuous alkylation and regeneration processes as per U.S. Pat. No. 7,176,340, US 2002/198422 and EP 1485334, and rare earth (RE) exchanged solid acid catalysts, as taught in U.S. Patent Application Publication 2008/0183025.

Surprisingly, however, it has been discovered that the use of rare earth exchanged molecular sieves (e.g., Y-zeolites) in such solid acid alkylation catalysts, endowed with a unique porosity distribution, can provide much higher activity and stability when compared to like catalysts without the special porosity characteristics described herein. This was especially surprising since in the past (U.S. Pat. No. 6,855,856) it was found that molecular sieves without RE required a totally different porosity distribution.

Thus, in one embodiment of the invention there is provided a solid catalyst comprising a hydrogenation metal and a solid acid in the form of a rare earth exchanged molecular sieve, wherein the catalyst is at least characterized by a porosity of less than 0.20 ml/g in pores below 100 nm in diameter, and a total porosity of greater than 0.30 ml/g.

Another embodiment of the invention provides a process for the alkylation of hydrocarbons comprising contacting a saturated hydrocarbon feedstock and one or more olefins with a catalyst of this invention at alkylation process conditions.

These and still further embodiments, features and advantages of the invention shall be made even more apparent by the followed detailed description, including the appended figures and claims.

### BRIEF DESCRIPTION OF THE DRAWINGS

FIG. 1 is a graph of porosity distribution by pore size, for the particular catalyst embodiments of this invention and for the comparative catalyst which is not of the invention, fabricated pursuant to the Experimental section of this disclosure.

FIG. 2 is a graph of catalytic activity expressed in terms of olefin conversion (as further defined below) over time for catalyst embodiments of the invention and a comparative catalyst which is not of the invention, fabricated pursuant to the Experimental section of this disclosure.

### FURTHER DETAILED DESCRIPTION OF THE INVENTION

The catalyst of this invention comprises a hydrogenation metal and a solid acid in the form of a rare earth exchanged molecular sieve. Examples of suitable hydrogenation metals are the transition metals, such as metals of Group VIII of the Periodic Table, and mixtures thereof. Among these, noble metals of Group VIII of the Periodic Table are preferred. Platinum is especially preferred. The amount of hydrogenation metal will depend on its nature. When the hydrogenation metal is a noble metal of Group VIII of the Periodic Table, the catalyst generally will contain in the range of about 0.01 to about 2 wt % of the metal, calculated as metal. In another embodiment the metal amount ranges from about 0.1 to about 1 wt %. Unless otherwise specified herein, weight percentages provided in this disclosure are based on the total weight of the dry catalyst, which can be calculated using the weight loss upon heating the catalyst for one hour at 600° C. (Loss on Ignition, or LOI 600, 1 hour)

Examples of molecular sieves are zeolites such as zeolite beta, MCM-22, MCM-36, mordenite, faujasites such as X-zeolites and Y-zeolites, including HY-zeolites and USY-zeolites. Preferred solid acids are zeolites, including, zeolite beta, faujasites such as X-zeolites and Y-zeolites, including HY-zeolites and USY-zeolites. Mixtures of solid acids can also be employed. In one embodiment the solid acid is a faujasite with a unit cell size (a₀) of 24.72 to about 25.00 angstroms, in another embodiment the solid acid is Y-zeolite with a unit cell size of 24.34-24.72 angstroms. In yet another embodiment the solid acid is Y-zeolite with a unit cell size of 24.56-24.72 angstroms.

The solid acid component of the catalyst comprises rare earth (RE), i.e., one or more elements chosen from the lanthanide series. In one embodiment, the rare earth amount ranges from about 0.5 wt % to about 32 wt %. In another, rare earth ranges from about 2 wt % to about 9 wt %. In yet another, rare earth ranges from about 3 wt % to about 6 wt %. All references herein to rare earth wt % are calculated as rare earth oxides on a dry basis (600° C., 1 hour). Lanthanum or lanthanum rich RE mixtures can be particularly suitable for use as the rare earth element(s). By lanthanum rich RE mixture it is meant that lanthanum would be about 70 to 80 wt % or more of the total amount of rare earth element(s) employed.

The rare earth element(s) may be exchanged into the solid acid component by conventional means described more fully below. During the exchange process of the solid acid component sodium (Na⁺) is removed from the catalyst. In one embodiment the solid acid component contains no more than about 1.5 wt % Na₂O; in another, no more than about 1.0 wt % Na₂O; and in yet another, less than or equal to about 0.8 wt % Na₂O. In still another embodiment, it contains less than or equal to about 0.6 wt % Na₂O, all calculated on a dry basis (600° C., 1 hour).

Certain catalysts of this invention can additionally comprise a matrix material. Examples of suitable matrix materials are alumina, silica, titanic, zirconia, clays, and mixtures thereof. Matrix materials comprising alumina are generally preferred. In one embodiment, the catalyst comprises about 10 wt % to about 40 wt % of the matrix material and balance solid acid, based on the total weight of the solid acid and the matrix material contained in the catalyst.

The catalyst preferably contains no halogen component.

Preferably, in addition to the hydrogenation metal component, the catalyst of the invention comprises about 65 to about 85 wt % of the solid acid and about 15 to about 35 wt % of the matrix material. More preferably, the catalyst comprises about 70 to about 80 wt % of the solid acid and about 20 to about 30 wt % of the matrix material.

The catalyst used in the process according to the invention is prepared by adjusting the water content. For example, the solid acid constituent may be mixed with a matrix material, to form carrier particles, followed by calcination of the particles. The hydrogenating function may, e.g., be incorporated into the catalyst composition by impregnating the carrier particles with a solution of a hydrogenation metal component. After impregnation the catalyst may be calcined.

In one embodiment, the catalyst is reduced at a temperature in the range of about 200 to about 500° C. in a reducing gas such as hydrogen. In another embodiment, the catalyst is reduced at a temperature in the range of about 250 to about 350° C. The reduction can be performed before adjustment of the water content, after addition of water to the catalyst and/or by using reduction as a way to adjust the water content. In one embodiment, the reduction is performed before adjustment of the water content. In another, the reduction is performed after drying the catalyst in a dry, non-reducing gas (such as nitrogen, helium, air, and the like).

The catalyst should contain an amount of water in the range of about 1.5 to about 6 wt %, while in another embodiment the water content is in the range of about 1.8 to about 4 wt %, and in another embodiment it is in the range of about 2 to about 4 wt %. The water content is defined as the water content during use in the alkylation process and is measured by determining the weight loss upon heating the catalyst for two hours at 600° C. (LOI). The water content of the catalyst can be adjusted by various methods as described in PCT/EP2005/000929, which is incorporated by reference in its entirety. Such methods are exemplified below as methods 1, 2, and 3.

Method 1 involves increasing the LOT of a catalyst by exposing the catalyst to water. This can be achieved by exposing the catalyst to a water-containing atmosphere, e.g., air at ambient conditions. Embodiments of this method include exposing a reduced catalyst to water until the desired LOT is reached, exposing an unreduced catalyst to water until an LOI above the desired level is reached, followed by reduction of the catalyst, thereby decreasing the LOT to the desired level, exposing a reduced catalyst to water until an LOT above the desired level is reached, followed by treatment of the catalyst in either an inert or a reducing atmosphere, thereby decreasing the LOT to the desired level, and reducing the catalyst in a hydrogen and water-containing atmosphere.

Method 2 involves decreasing the LOT of an existing catalyst to the desired level by reducing an unreduced catalyst with an LOT above the desired level.

Method 3 involves in-situ water addition by starting the alkylation process with a catalyst having an LOT below the desired level and adding water to the alkylation unit during processing, for instance by adding water to the hydrocarbon feed, by regenerating the catalyst in a water-containing atmosphere and/or by exposing the regenerated catalyst to a water-containing atmosphere.

A combination of two or more of the above methods may also be employed.

Preferably, the catalyst consists essentially of a hydrogenation metal, a rare earth exchanged molecular sieve and, optionally, a matrix material. More preferably, the catalyst consists essentially of one or more rare earth exchanged faujasite(s), one or more Group VIII noble metal(s), and one or more matrix material(s). Even more preferably, the catalyst of the invention consists essentially of one or more Group VIII noble metal compounds, one or more rare earth exchanged Y-zeolites, and one or more matrices comprising alumina.

The catalyst can be prepared by processes now known to the industry, modified to achieve the particular pore characteristics of this invention. A typical process comprises the successive steps of

(i) shaping, e.g., extruding the solid acid constituent, optionally after mixing it with a matrix material, to form particles,
(ii) calcining the resulting particles, and
(iii) incorporating the hydrogenation metal into the calcined particles by, e.g., impregnating the particles with a solution of a hydrogenation metal component and/or by (competitive) ion exchange.
Alternatively, the catalyst can, e.g., be prepared by a process comprising the successive steps of
(i) incorporating the hydrogenation metal into the solid acid constituent or into a mixture of the solid acid constituent and the matrix material,
(ii) shaping, e.g., extruding the resulting material to form particles, and
(iii) calcining the resulting particles.
With regard to catalyst preparation, the procedures described in US 2008183025 also can be followed. In order to obtain the particular porosity characteristics of the present invention, it is particularly useful to carry out the extrusion step carefully. Thus, it is particularly useful to carry out the extrusion as follows:
1) mixing the matrix material (e.g., precipitated alumina powder), rare earth-exchanged molecular sieve (e.g., zeolite), water, nitric acid and a few percent of an extrusion aid (e.g. methylcellulose) to form a mixture,
2) feeding this mixture to an extruder, and
3) depending on visual inspection of the resulting extrusion product, adding some extra water during extrusion.
In carrying out this procedure experimentally to obtain catalysts of the invention, it was observed that water content (LOI 600° C., 1 hour) of the final extrusion mixture was in the order of 40 to 45 wt %. In the order of 0.15 to 0.25 equivalent (relative to the alumina powder) of nitric acid was added. Zeolite content of the extrudates was in the order of 65 to 85 wt % and the balance matrix and hydrogenation metal (0.05 to 0.5 wt % Pt), calculated on dry basis (600° C., 1 hour). Those skilled in the art can now appreciate that the exact LOT and acid addition required to get the extrudates with the desired properties (including physical strength such as side crushing strength and bulk crushing strength) depend on the molecular sieve content and the specific properties of the matrix material used. This is typically found by trial and error experiments after the starting component materials have been determined. The average particle length ranges from about 2 to about 6 mm, the particle diameter ranges from about 0.5 to about 3 mm, and the side crushing strength ranges from about 1.5 to about 10 lbs/mm.

The catalyst is particularly suitable for the alkylation of saturated hydrocarbons. The invention therefore further pertains to the use of the catalyst of the invention in the alkylation of these feedstocks. As stated above, this comprises the reaction of a saturated hydrocarbon with an olefin or olefin precursor in the presence of the catalyst of the invention to give highly branched saturated hydrocarbons with a higher molecular weight.

Preferably, the hydrocarbon is a branched saturated hydrocarbon such as an isoalkane having about 4-10 carbon atoms. Examples of suitable isoalkanes are isobutane, isopentane, isohexane or mixtures thereof, with isobutane being most preferred. The olefins to be used in the alkylation process generally have about 2-10 carbon atoms, preferably 2-6 carbon atoms, still more preferably about 3-5 carbon atoms, and most preferably about 4 carbon atoms. Most preferably, the alkylation process consists of the alkylation of isobutane with butenes.

As will be evident to the skilled person, the alkylation process can be applied in any suitable form, including fluidized bed processes, slurry processes, and fixed bed processes. The process may be carried out in a number of beds and/or reactors, each with separate olefin addition. In such a case, the process of the invention may be carried out in each separate bed or reactor.

Suitable alkylation process conditions are known to the skilled person. Preferably, an alkylation process as disclosed in WO 9823560 is applied, but using the catalyst herein described. The process conditions applied in this process are summarized in the following Table:

| | Temp. | Pressure | Molar ratio of saturated |
|---|---|---|---|
| | Range (° C.) | Range (bar) | hydrocarbon to olefin |
| Preferred | −40-250 | 1-100 | 5:1-5000:1 |
| More preferred | 20-150 | 5-40 | 50:1-1000:1 |
| Most preferred | 65-95 | 15-30 | 150:1-750:1 |

Preferably, a regeneration technique as described in WO 9823560 is applied during the alkylation process. More in particular, during the alkylation process the catalyst is preferably subjected intermittently to a regeneration step by being contacted with a feed containing an aliphatic compound and hydrogen, with said regeneration preferably being carried out at about 90% or less, more preferably at about 60% or less, even more preferably at about 20% or less, and most preferably at about 10% or less of the active cycle of the catalyst. The active cycle of the catalyst is defined as the time from the start of the feeding of the alkylation agent to the moment when, in comparison with the entrance of the catalyst-containing reactor section, about 20% of the alkylation agent leaves the catalyst-containing reactor section without being converted, not counting isomerisation inside the molecule.

Optionally, in this process, the catalyst can be subjected periodically to a high-temperature regeneration with hydrogen in the gas phase. This high-temperature regeneration is preferably carried out at a temperature of at least about 150° C., more preferably at about 175-600° C., and most preferably at about 200-400° C. For details of this regeneration procedure, reference is made to WO 9823560, and in particular to page 4, lines 5-19 and page 9, line 13 through page 13, line 2. The present inventive catalyst may be used in batch, semi-continuous and continuous alkylation processes, and may undergo regeneration. Thus, the alkylation processes taught, e.g., in WO/9823560 (U.S. Pat. No. 5,986,158), US Patent Application Publication 2007/0293390, WO 2005/075387, U.S. Pat. No. 7,176,340, US 2002/198422 and EP 1485334, and U.S. Patent Application Publication 2008/0183025, can be carried out using the present catalyst under conditions taught therein.

The use of the catalyst of the present invention in the above alkylation process results in a high olefin conversion (amount of olefin in the feed that is converted in the reaction), a high C5+ alkylate yield (weight amount of C5+ alkylate produced divided by the overall weight of olefin consumed) and a high octane number, while the amount of undesired C9+ by-products can be restricted and the catalyst's stability can thus be improved. For details in respect of these parameters, reference is made to WO 9823560.

The following examples are presented for purposes of illustration, and are not intended to impose limitations on the scope of this invention.

### EXPERIMENTAL

The extruder used in the experiments was a commercially available twin screw extruder from Werner-Pfleiderer Corp., model number ZSK-30. In addition, the pore volume for pores less than 100 nm in diameter, as well as the total pore volume of produced catalysts were determined via mercury (Hg) intrusion on the basis of the Washburn equation

$$D = \frac{- 4 γ cos θ}{p}$$

with D being the pore diameter, p being the pressure applied during the measurement, γ being the surface tension, taken to be 480 dynes/cm, and θ being the contact angle, taken to be 140°. In the present measurement, the pressure was varied over such a range that the measurement covered pores with a diameter in the range of 3.6-8000 nm.

In these experimental samples, about 70 to about 83 wt % of rare earth exchanged Y-zeolite was used in making each catalyst, with the balance being alumina matrix in the samples prior to extrusion.

The Y-zeolite with rare earth ions had been prepared via a route described in US 2008183025, i.e., sodium-Y-zeolite (NaY) was prepared (silica to alumina molar ratio (SAR) 5.5, Na₂O about 13 wt %) followed by ion exchange with rare earth ions (preferably a lanthanum rich RE mixture) and NH₄+-ions (remaining Na₂O typically about 4.2 wt %) and steaming at about 400 to about 500° C. After the steam treatment, exchange with NH₄⁺-ions is carried out and then the zeolite is dried. However, multiple steaming and ion exchange with NH₄⁺-ions steps may be employed if required to achieve appropriate SAR, a₀ and Na₂O content.

The tested catalysts contained about 0.20 wt % platinum by impregnation of calcined extrudates, and the zeolite Na₂O content was about 0.8 wt %, the zeolite a₀ was about 24.66 and RE content was about 4 wt %. Zeolite content varied in the samples between 70 and 75 wt %. As will be seen from the data presented here, the activity of the sample with the highest activity was much higher (>20%) than can be explained from the difference in zeolite content of less than 10% (75% vs. 70% zeolite). By varying the amount of matrix and zeolite and by addition of acid (e.g., HNO₃) and water the porosity of catalyst particles formed by shaping techniques such as extrusion can be controlled. The size of the zeolite particles as measured by scanning electron microcopy (SEM) was in the order of 100 to 1000 nm.

Catalysts A-D were prepared in accordance with the following procedure; In the case of catalyst C and D 70% and in the case of catalyst A and B 75% of the RE exchanged zeolite prepared as above-described was used and the balance alumina matrix and about 0.20 wt % Pt (all calculated on a dry basis LOI 600° C. 1 hour). Extrusion was carried out as mentioned above. Average length of the extrudates was about 4 mm and the average diameter was about 1 mm. Consequently the specific length calculated according to the methods referred to before in U.S. Pat. No. 6,855,856 was about 0.22 mm.

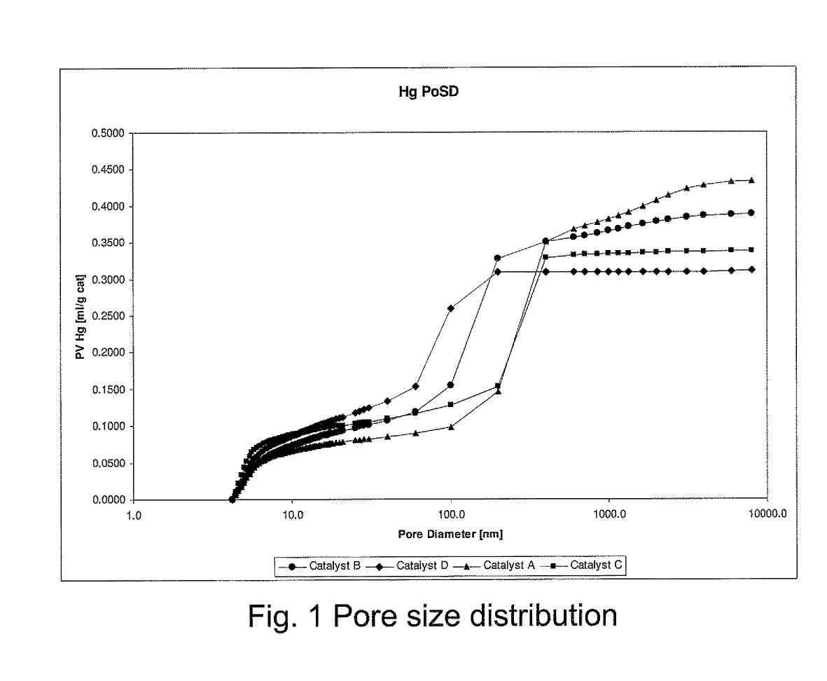

Each catalyst was analyzed for pore volume using the Hg method referenced above. The pore volume distribution is graphed in FIG. 1, and determined pore volumes in pores with diameters of less than 100 nm and total pore volume are set out in Table I below.

TABLE 1

| | Hg Pore Volume in <100 | Hg Total Pore |
|---|---|---|
| Catalyst | nm Pores (ml/g) | Volume (ml/g) |
| A | 0.10 | 0.43 |
| B | 0.16 | 0.39 |
| C | 0.13 | 0.34 |
| D | 0.26 | 0.31 |

In table 2 macroporosity as defined in U.S. Pat. No. 6,855,856 and the ratio of macroporosity and specific length are presented for catalysts A-D.

TABLE 2

| | Macropore (>40 nm) | Macropore (>40 nm) Volume Divided |
|---|---|---|
| Catalyst | Volume (ml/g) | by Specific Length of 0.22 |
| A | 0.35 | 1.59 |
| B | 0.28 | 1.27 |
| C | 0.22 | 1 |
| D | 0.18 | 0.82 |

It can be seen that only catalyst D (the reference) has the properties preferred according to U.S. Pat. No. 6,855,856. The catalyst A, B and C of the current invention show a distinct difference compared to the preferred properties of the earlier invention illustrating the surprising behavior of the catalysts of the current invention. It will be noted that macroporosity (pores>40 nm) of catalysts ATC is relatively high compared to that of catalyst D.

The specific length of catalyst A was 0.22 mm and macropore volume estimated from the mercury intrusion measurements (see e.g. graph of FIG. 1 was about 0.36 ml/g, so that the ratio of macropore volume to specific length is 0.36/0.22=1.6, much higher than the maximum ratio previously taught in U.S. Pat. No. 6,855,856.

Each catalyst A-D was used in an alkylation process carried out as follows: A fixed-bed recycle reactor as described in WO 9823560, which is herein incorporated by reference in its entirety, having a diameter of 2 cm was filled with a 1:1 volume/volume mixture of 38.6 grams of catalyst extrudates (on dry basis, i.e. the actual weight corrected for the water content) and carborundum particles (60 mesh). At the center of the reactor tube a thermocouple of 6 mm in diameter was arranged. The reactor was flushed with dry nitrogen for 30 minutes (21 Nl/hour). Next, the system was tested for leakages at elevated pressure, after which the pressure was set to 21 bar and the nitrogen flow to 21 Nl/hour. The reactor temperature was then raised to 275° C. at a rate of 1° C./min, at 275° C. nitrogen was replaced by dry hydrogen and the catalyst was reduced at 275° C.

Alternatively, in ease of high temperature regeneration of the same catalyst sample between runs, after draining and flushing the reactor with hydrogen to remove hydrocarbons while maintaining the alkylation reaction temperature, hydrogen flow was set to 21 Nl/hour and the reactor temperature was then raised to 275° C. at a rate of 1° C./min, and the catalyst was regenerated at 275° C.

After 2 hours, the reactor temperature was lowered to the reaction temperature of about 75° C. During cooling down water was added to the hydrogen flow to obtain an LOT of the catalyst of about 2-4 wt % (in this case the LOI of the catalyst is defined as the catalyst's weight loss after heating for two hours at 600° C.).

The hydrogen stream was stopped upon attaining the reaction temperature. Isobutane containing about 4 wt % alkylate (added to accelerate deactivation rate, composition of the alkylate added is similar to alkylate produced by the process at the conditions described) and about 1 mol % of dissolved hydrogen was supplied to the reactor at a rate of about 4.0 kg/hour. About 95-98% of the isobutane/alkylate mixture was fed back to the reactor. About 2-5% was drained off for analysis. Such an amount of isobutane/alkylate mixture was supplied to the reactor to ensure a constant quantity of liquid in the system. When the system had stabilized, hydrogen addition was stopped and such an amount of cis-2-butene was added to it as to give a cis-2-butene-WHSV of 0.16. The overall rate of flow of liquid in the system was maintained at about 4.0 kg/h. The weight ratio of isobutane to cis-2-butene at the reactor inlet was about 500-650. The pressure in the reactor amounted to about 21 bar. Total alkylate concentration of the hydrocarbon recycle flow (from added and produced alkylate) was maintained at about 10 wt % during the test by controlling the drain off flow to analyses.

Each time after 1 hour of reaction, the catalyst was regenerated by being washed with isobutane/alkylate mixture for 5 minutes, followed by 50 minutes of regeneration through being contacted with a solution of 1 mole % of H₂ in isobutane/alkylate mixture, and then being washed with isobutane/alkylate mixture for another 5 minutes (total washing and regeneration time 1 hour). After this washing step, alkylation was started again.

The temperature during the washing steps, the regeneration step, and the reaction step was the same.

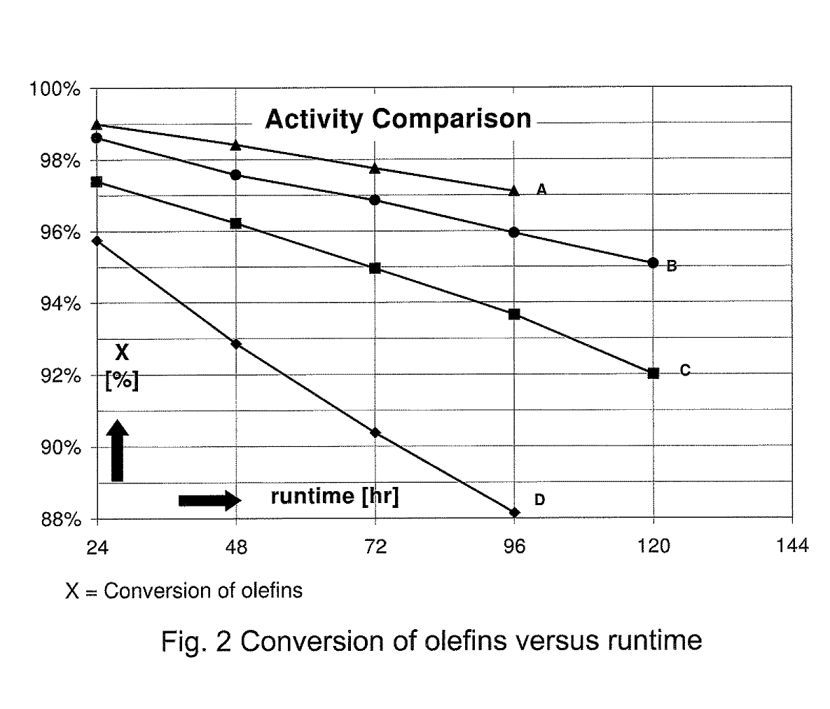

The process was conducted as above and the catalytic performance was measured as a function of time. The performance was characterized by the olefin conversion per reactor pass. Olefin conversion per reactor pass is the weight fraction (as a percentage) of olefins that is converted between the inlet—and the outlet of the catalyst bed, not counting isomerization within the olefin molecules. The results are plotted in the graph of FIG. 2.

As can be seen from the graphed results of the catalytic activity of Catalysts A through D in FIG. 2, the catalysts having the combination of a pore volume less than 0.2 ml/g in pores smaller than 100 nm in diameter and a total pore volume greater than 0.3 ml/g (Catalysts A, B and C) showed surprisingly beneficial results when compared to the performance of Catalyst D. The advantageous increase in activity of catalysts A-C shown in FIG. 2 cannot be explained away on a percentage basis by the percentage of increase in the amount of zeolite in the catalysts A, B and C versus that of catalyst D. This unique combination of pore characteristics appear to provide an unexpectedly beneficial improvement in alkylation activity.

It is to be understood that the reactants and components referred to by chemical name or formula anywhere in this document, whether referred to in the singular or plural, are identified as they exist prior to coming into contact with another substance referred to by chemical name or chemical type (e.g., another reactant, a solvent, or etc.). It matters not what preliminary chemical changes, transformations and/or reactions, if any, take place in the resulting mixture or solution or reaction medium as such changes, transformations and/or reactions are the natural result of bringing the specified reactants and/or components together under the conditions called for pursuant to this disclosure. Thus the reactants and components are identified as ingredients to be brought together in connection with performing a desired chemical operation or reaction or in forming a mixture to be used in conducting a desired operation or reaction. Also, even though an embodiment may refer to substances, components and/or ingredients in the present tense (“is comprised of”, “comprises”, “is”, etc.), the reference is to the substance, component or ingredient as it existed at the time just before it was first contacted, blended or mixed with one or more other substances, components and/or ingredients in accordance with the present disclosure.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

Each and every patent or other publication or published document referred to in any portion of this specification is incorporated in toto into this disclosure by reference, as if fully set forth herein. Any inconsistency between a cited document incorporated herein by reference and the explicit text of this disclosure should be resolved in favor of the explicit text of this disclosure.

This invention is susceptible to considerable variation within the spirit and scope of the appended claims.

1: A solid catalyst comprising a hydrogenation metal and a solid acid in the form of a rare earth exchanged molecular sieve, wherein the catalyst is at least characterized by a porosity of less than 0.20 ml/g in pores below 100 nm in diameter, and a total porosity of greater than 0.30 ml/g.

2: The solid acid catalyst according to claim 1 wherein the molecular sieve comprises a zeolite.

3: The solid acid catalyst according to claim 2 wherein the zeolite comprises a zeolite having a faujasite structure.

4: The solid acid catalyst according to claim 3 wherein the zeolite is Y-zeolite.

5: The solid acid catalyst according to claim 4, wherein the Y-zeolite has a unit cell size in the range of 24.56-24.72 angstroms.

6: The solid acid catalyst according to claim 5, wherein the unit cell size is in the range of 24.62-24.70 angstroms.

7: The solid acid catalyst according to claim 1, wherein the solid acid comprises no more than about 1 wt % Na₂O, calculated on a dry basis (600° C., 1 hour).

8: The solid acid catalyst according to claim 4, wherein the solid acid comprises no more than about 0.8 wt % Na₂O calculated on a dry basis (600° C., 1 hour).

9: The solid catalyst according to claim 1, wherein the porosity in pores below 100 nm in diameter is less than 0.18 ml/g and the total porosity is more than 0.30 ml/g.

10: The solid catalyst according to claim 8, wherein the porosity in pores below 100 nm in diameter is less than 0.18 ml/g and the total porosity is more than 0.34 ml/g.

11: The solid catalyst according to claim 1, wherein the hydrogenation metal consists essentially of a Group VIII noble metal.

12: The solid acid catalyst according to claim 11, wherein the Group VIII noble metal is platinum.

13: The solid acid catalyst according to claim 1, wherein the catalyst additionally comprises a matrix material.

14: The solid acid catalyst according to claim 13, wherein the matrix material comprises alumina.

15: The solid acid catalyst according to claim 1, wherein the rare earth is lanthanum or a lanthanum rich mixture of rare earth elements.

16: The solid acid catalyst according to claim 1, wherein the catalyst further comprises an amount of water in the range of about 1.5 to about 6 wt %.

17: The solid acid catalyst according to claim 16, wherein the amount of water is in the range of about 2 to about 4 wt %.

18: A process for the alkylation of hydrocarbons comprising contacting a saturated hydrocarbon feedstock and olefins with a catalyst at alkylation process conditions, the catalyst being in accordance claim 1.
